(19) United States
(12) Patent Application Publication
(10) Pub. No.: US 2024/0189550 A1
(43) Pub. Date: Jun. 13, 2024

(54) SAFETY CANNULA ASSEMBLY

(71) Applicant: SARSTEDT AG & CO. KG, Nuembrecht (DE)

(72) Inventor: Viktor HAUPT, Nuembrecht (DE)

(21) Appl. No.: 18/542,621

(22) Filed: Dec. 16, 2023

Related U.S. Application Data
(63) Continuation of application No. PCT/EP2022/065999, filed on Jun. 13, 2022.

(30) Foreign Application Priority Data
Jun. 16, 2021 (DE) DE102021115561.4

(51) Int. Cl. A61M 25/06; A61M 39/02

(52) U.S. Cl. CPC A61M 25/0625; A61M 25/0637; A61M 39/0247; A61M 2039/0258

(57) ABSTRACT

A safety cannula assembly, comprising: a cannula configured to puncture human or animal tissue, wherein the cannula includes a distal end portion including a tip; a sliding body including a distal end portion connected to the cannula and a proximal end portion connected to a flexible tube, wherein a flow connection extends through the sliding body between the tip of the cannula and a proximal end of the flexible tube; a main body in which the sliding body is displaceable from an operating position, where the tip is located outside the main body to a safety position, where the tip is located inside the main body, wherein a stop surface of the sliding body contacts a stop surface of the main body in the safety position, so that further axial displacement of the sliding body relative to the main body in the proximal direction is prevented.

### RELATED APPLICATIONS

This application is a continuation of International patent application PCT/EP2022/065999 filed on Jun. 13, 2022 claiming priority from German Patent application DE 10 2021 115 561.4 filed on Jun. 16, 2021, both of which are incorporated in their entirely by this reference.

### FIELD OF THE INVENTION

The invention relates to a safety cannula assembly.

A positional or directional indication “proximal” according to the invention indicates an arrangement oriented towards a body of a person using the safety cannula assembly, in particular an arrangement or position oriented towards a hand of the person holding the safety cannula arrangement, whereas “distal” indicates an arrangement oriented away from the body or the hand. Consequently a proximal end of an object, thus also of the safety cannula arrangement according to the invention is closer to the body of the person than a distal end.

Cannula arrangements according to the invention are typically used in medical applications to draw a bodily fluid, in particular venous blood from a patient or to feed a fluid into a fluid vessel of the human body. Safety cannula assemblies are used to prevent injuries from needle punctures after use in an optimum manner and to provide maximum comfort for the person using the cannula assembly and to prevent pain and trauma for the patient using the cannula arrangement including activating the needle protection. A pull back movement of the needle after completion of the blood draw or infusion is performed automatically for a passive needle protection without further interaction by the user, whereas an active needle protection requires an intentional action by the person using the cannula arrangement to move the cannula into the safety position. The instant invention primarily relates to safety cannula assemblies with active needle protection.

The main body according to the invention is defined as performing a housing function and having an enveloped interior space that includes at least a portion of the sliding body in the operating position, wherein at least the cannula itself including its tip is housed in the interior space of the sliding body in the safety position, this means after displacing the sliding body, in order to prevent puncture wounds from the tip and also contact with the needle where contaminated or infectious body substance might be arranged. The main body may have a single part configuration or a multi-part configuration according to the invention wherein the individual main body parts can be inserted or clipped into one another or connected with one another in case of the multi-part configuration. Also, the connection of plural main body parts with so called film hinges is conceivable in the context of the instant invention. Walls of the main body do not have to be completely closed in this context but can also have cut outs, interruptions, slots, or other perforations so that a hermetic sealing of the interior towards the ambient does not have to be provided.

An actuation element according to the instant invention is an element configured to impart a required force upon a sliding body functioning as a drive to move the sliding body from the operating position into the safety position. A drive element and an associated energy storage device can be separate from each other. However, the two functions can be combined in one element, in particular configured as spring element that either stores mechanical energy in the form of a coil spring and/or pressure energy like e.g. in a preloaded gas storage device.

A sliding body according to this application is an element which receives the cannula at a distal end and which is connected with a tube at a proximal end. The sliding body is typically also designated as cannula holder or needle carrier.

### BACKGROUND OF THE INVENTION

EP 3 466 464 A1 discloses a needle assembly where the proximal end of the main body as well as the proximal end of the sliding body are significantly thickened with an oval cross section compared to sections extending further in the distal direction. These thickened end sections include spring elastic tongues that are arranged diametrically opposed at the sliding body, wherein free ends of the tongues are configured as locking elements whereas the main body includes recesses in the thickened portion wherein the recesses cooperate with locking elements at the sliding body and received the locking ends in the operating position. In the interlocking condition, this means in the operating position of the needle arrangement the interlocking connection between the locking elements and the associated recesses in the main body prevent a rotation of the sliding body and thus also a rotation of the cannula about its longitudinal axis. This is important so that the tapered surface of the cannula tip has a precisely defined arrangement which is very important for the user to be able to perform a correct puncture.

After using the known needle arrangement and the activating the needle protection the sliding body with the cannula is displaced relative to the main body in the proximal direction. An end of the pull back movement is caused by a stop surface at a shoulder proximally arranged at the cannula carrier wherein the shoulder cooperates with a radially inward extending shoulder at the inner enveloping surface of the main body. A similar functional principle with respect to the rotation safety is also disclosed in WO 2015 058 402 A1.

Additionally WO 2017/033449 describes a cannula arrangement including two trigger members pivotably arranged at an outer circumference of the main body. The trigger members are arranged like a rocker, this means pressure upon the actual trigger surface moves a locking element radially outward at an opposite end of the rocker. The locking elements of the trigger members engage faces of locking elements configured as two arms protruding radially outward from the sliding body in the operating position of the cannula arrangement, but depressing the trigger surfaces of the rockers disengages the engagement between the rocker and the arm so that the spring-loaded cannula carrier including the tube arranged thereon are moved backward. FIG. 4 shows the operating position of the cannula with engaged locking elements. FIG. 5 shows a subsequent intermediary position with a cannula carrier that is slightly displaced backward and the safety position of the cannula carrier is shown in FIG. 6 of the document cited supra.

It is evident from FIGS. 1 and 2 of the prior art document recited supra that the cut outs for the arms do not extend circumferentially in the main body but that the cut outs have the shape of two discrete windows respectively configured with walls there between. Therefore the arms also function as rotation safety since they respectively engage a window shaped opening whose walls function as stops for the arms in the circumferential direction and prevent their rotation.

The arms of the WO 2017/033449 have an additional function to form a stop surface for the sliding body in the safety position which is evident from FIG. 6 which shows an engagement of the arms in a window respectively arranged at a proximal end of the main body. A wall arranged towards the distal end of the window contacts the arms due to the preload so that a stop is formed. The described double function of the arms which provides a rotation blocking of the sliding body in the operating position and also a stop of the sliding body in the safety position requires an elastic deformability of the arms in order to be able to reduce the radial dimension of the sliding body starting from the operating position and subsequently increase the radial dimension of the sliding body again to achieve the blocking function in the safety position.

WO 2019/177674 additionally discloses a holder for a blood drawing vial including a single needle arranged at a distal end. The sliding body shown in FIGS. 11 and 20 of this document and provided with the cannula includes a square flange adjacent to its proximal end wherein the square flange functions as a rotation blocking body. However, the axial displacement of the sliding body is not performed within the main body that is configured hollow cylindrical and used for receiving the blood drawing vial. Since a movement of a separate pull back body transversal to the direction or the axis occurs in order to perform the pull back movement of the sliding body, the sliding body then moves in the separate pull back body which has an axial offset from the receiving cylinder for the blood drawing vial. Therefore no tube is provided at the end of the sliding body according to WO2019/177674 A1, but the cannula runs through the sliding body and exits with a free cannula section at the proximal end of the sliding body wherein the free cannula section is used for puncturing a closure of the blood drawing vial.

WO2016/007438 A1 describes a needle arrangement with a retractable needle and septum. The needle carrier includes a spring elastic tongue which protrudes through a window in the main body with an inclined trigger surface and is thus reachable for an operator with a finger and loadable with a radially inward oriented pressure force. A shoulder that functions as a locking element is arranged at the spring elastic tongue and cooperates with a wall forming an opposite surface at a proximal end of the window in the main body and retains the preloaded sliding body in the operating position. Since the window in the main body with its walls running parallel to the longitudinal axis of the needle carrier is arranged at a small distance to the walls of the spring elastic tongue of the sliding body running parallel thereto, a rotation of the sliding body in the operating position is prevented. A triggering of the pull back mechanism by depressing the spring elastic tongue is performed after use of the needle arrangement so that the spring elastic tongue penetrates in its entirety into the window and is subsequently displaced in the proximal direction in the depressed condition axially within the main body. An end stop of the sliding body is formed on the one hand side by a radially inward protruding shoulder at the proximal end of the main body and on the other hand side by a radially outward protruding shoulder at the sliding body.

Furthermore EP 1 479 408 A1 discloses a safety cannula arrangement where locking elements engage one another in a portion of thickened proximal ends of the sliding body and of the main body. Pressure upon the opposite tongues that are arranged at the sliding body facilitates activating the pull back mechanism for the cannula. An end stop for the pulled back sliding body is implemented by preloaded spreading arms at the distal end of the sliding body and entering an undercut groove where the locking elements previously engaged the tongues of the sliding body. A safety against a rotation of the sliding body in the operating position of the known safety cannula arrangement is achieved in that the sliding body is provided with grooves that run in the direction of the longitudinal axis and that have a cross section of a circular segment, whereas the main body has corresponding circular segment shaped ribs that extend in the direction of the longitudinal axis of the sliding body.

### BRIEF SUMMARY OF THE INVENTION

Thus, it is an object of the invention to propose a safety cannula arrangement that is characterized by low complexity of the main body and of the cannula carrier with respect to implementing the functions of rotation blocking and end stop.

Improving upon the generic safety cannula arrangement recited supra the object is achieved A safety cannula assembly, comprising a cannula configured to puncture human or animal tissue, wherein the cannula includes a distal end portion including a tip; a sliding body including a distal end portion connected to the cannula and a proximal end portion connected to a flexible tube, wherein a flow connection extends through the sliding body between the tip of the cannula and a proximal end of the flexible tube; a main body in which the sliding body is displaceable from an operating position, where the tip of the cannula is located outside the main body to a safety position, where the tip of the cannula is located inside the main body, wherein a stop surface of the sliding body contacts a stop surface of the main body in the safety position, so that further axial displacement of the sliding body relative to the main body in the proximal direction is prevented; an actuation element arranged between the main body and the sliding body and configured to displace the sliding body from the operating position to the safety position; and a trigger mechanism arranged at the main body and configured to trigger the displacement of the sliding body from the operating position to the safety position, wherein the trigger mechanism includes at least one trigger member loadable by a radially oriented compressive force or loadable with a torque relative to the main body about a longitudinal axis of the main body, and a locking element displaceable from a locking position, where the locking element engages the sliding body and locks the sliding body in the operating position, to a trigger position, where the locking element is disengaged from the sliding body so that the sliding body moves into the safety position; a rotation blocking device which prevents a rotation of the sliding body arranged in the operating position about its longitudinal axis inside the main body, wherein the rotation blocking device includes at least one rotation blocking body of the sliding body and at least one rotation blocking body of the main body that cooperates with the at least one rotation blocking body of the sliding body, wherein the stop surface of the sliding body is arranged at the at least one rotation blocking body of the sliding body.

The sliding body according to the invention is thus characterized by a simple configuration and ease of production. In particular, the invention is advantageously useable where the locking elements fixing the sliding body in the operating position are not simultaneously used as rotation blocking bodies. Many known safety cannula arrangements use this double function of the locking elements. According to the concept of the invention, the rotation blocking body can be formed by a separate component in addition to the locking elements of the trigger mechanism and can be typically arranged offset, advantageously axially offset from the locking elements. The rotation blocking body and the stop surface of the sliding body, however, are formed by the same component or are formed at the same component so that the double function provides and advantageous embodiment with respect to installation space and complexity. The invention is flexible on how the rotation blocking is achieved by the rotation blocking body of the sliding body. All geometries that prevent a rotation of the sliding body about its longitudinal axis but facilitate an axial movability of the sliding body in the main body for transfer into the safety position are conceivable.

Advantageously the sliding body is configured rigid overall. This means that the sliding body is not elastically or plastically deformed under inner and outer forces and torques substantially or in a manner that is required for the function when the safety cannula arrangement is used as intended. This would not be the case for tongues that are integrally formed at the sliding body, spring elements which assume different positions or shapes in the operating position or the safety position or a position there between.

Additionally, the sliding body advantageously has the same shape and/or configuration in the operating position and in the safety position and in all intermediary positions arranged there between.

It is provided according to an advantageous embodiment of the invention that the main body is provided with a respective gripping lobe at two opposite sides so that handling is improved or an alternative handling method besides handling the actual main body is provided.

Ergonomics are further improved when a grip portion for handling the device during punction is advantageously exclusively arranged at the main body, wherein ribs or a plurality of protrusions improving gripping are advantageously arranged in the grip portion. This prevents in particular that the safety cannula arrangement also has to be handled in the portion of the cannula carrier which is disadvantageous because the cannula carrier moves relative to the main body when the trigger mechanism is actuated.

In an advantageous embodiment of the invention the sliding body is configured as a hollow cylinder and the rotation blocking body of the sliding body is a protrusion or flange that protrudes radially outward from an outer enveloping surface of the hollow cylinder and that advantageously has a square cross section viewed in the direction of the longitudinal axis of the sliding body, wherein corner portions of the square cross section are advantageously rounded. The square cross section of the circumference of the protrusion or flange has advantages with respect to assembling the safety cannula arrangement since it provides an exact positioning of the cannula tip relative to the main body in a simple manner for a known orientation of cannula tip relative to the sliding body. Advantageously a protrusion or flange with a square circumference cooperates with a square cross section in a section of an interior cavity of the main body to achieve the required rotation blocking. As a matter of principle the protrusion or flange can also have a polygonal or elliptical shape.

In addition to the double function of a rotation blocking and forming a stop surface the rotation blocking body arranged at the sliding body can also perform a third function. The adjustment element can be supported at a distal face of the rotation blocking body arranged at the sliding body, wherein the distal face is arranged opposite to a stop surface that limits the pull back movement. This way the configuration is particularly simple and cost effective.

According to an advantageous embodiment of the safety cannula arrangement according to the invention, the stop surface of the main body can be formed by at least one protrusion that extends radially inward from a wall defining the inner cavity of the main body. The protrusion can be formed e.g. by a shoulder in the main body wall or by a typically distal main body face wall which can also be a partially formed face wall.

Last not least a particularly advantageous embodiment includes two of the protrusions recited supra that are arranged diametrically opposed to one another and that are respectively formed as a triangle, in particular with an arcuate cambered contour, viewed in the direction of the longitudinal axis of the sliding body, wherein each protrusion advantageously connects two L-shaped wall sections of the main body that are arranged L-shaped in cross section. In an embodiment of the main body that has two parts in the portion of the rotation blocking device the protrusions functioning as stops also perform a stiffening function preventing an unintentional deformation of the wall sections that are connected with one another and arranged L-shaped so that maintaining the rectangular cross section of the main body in this area is facilitated.

### BRIEF DESCRIPTION OF THE DRAWINGS

The invention is subsequently described based on an embodiment with reference to drawing figures, wherein

FIGS. 1 and 2: illustrate a safety cannula arrangement in two perspective views including a cannula protection plugged on distally;

FIG. 3: illustrates an exploded view of the safety cannula arrangement;

FIG. 4: Illustrates a perspective view of the main body including the cannula protruding there from;

FIG. 5: is similar to FIG. 4 but shows a section of the main body;

FIG. 6: illustrates a released first main body part including a sliding body inserted therein;

FIG. 7: illustrates the sliding body with a cannula, actuation element and a section of a flexible tube;

FIG. 8: is similar to FIG. 7, but does not include the actuation element;

FIG. 9: illustrates the perspective view of the main body including a second and third main body part folded out laterally;

FIG. 10: illustrates a perspective view of the first and second main body part;

FIG. 10a: is similar to FIG. 10, but shows a different perspective;

FIGS. 11 and 12: illustrates a longitudinal sectional view according to FIGS. 1 and 2 showing the sliding body in the operating position;

FIG. 12a: illustrates a blown-up detail of FIG. 12

FIGS. 13 through 16: illustrate a sectional view along the line I-I through the safety cannula arrangement according to FIGS. 1 and 2 in different positions of the trigger members and locking elements;

FIG. 17: illustrates a perspective view of the safety needle in a sectional view along the line II-II in FIGS. 1 and 2;

FIG. 18: illustrates a trigger mechanism in a sectional view along the line III-III like in FIG. 1 looking in the proximal direction;

FIG. 19: illustrates a longitudinal sectional view through the safety needle arrangement according to FIGS. 1 and 2 in the safety position of the sliding body;

FIG. 19a: illustrates a blown-up detail of FIG. 19;

FIG. 20: Illustrates a sectional view of the safety cannula arrangement according to FIGS. 1 and 2 along the sectional like IV-IV with the sliding body in the safety position in the distal viewing direction;

FIG. 21: is similar to FIG. 18, however shows the safety position of the sliding body; and

FIG. 22: Illustrates a cross sectional view of the safety cannula arrangement according to FIGS. 1 and 2 along the line V-V in the distal viewing direction.

### DETAILED DESCRIPTION OF THE INVENTION

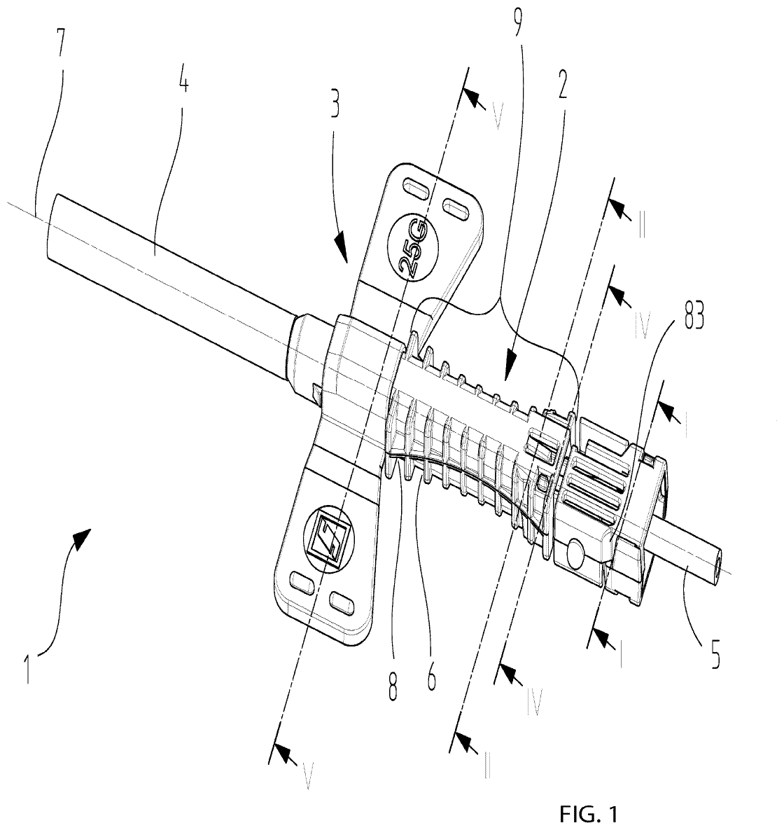

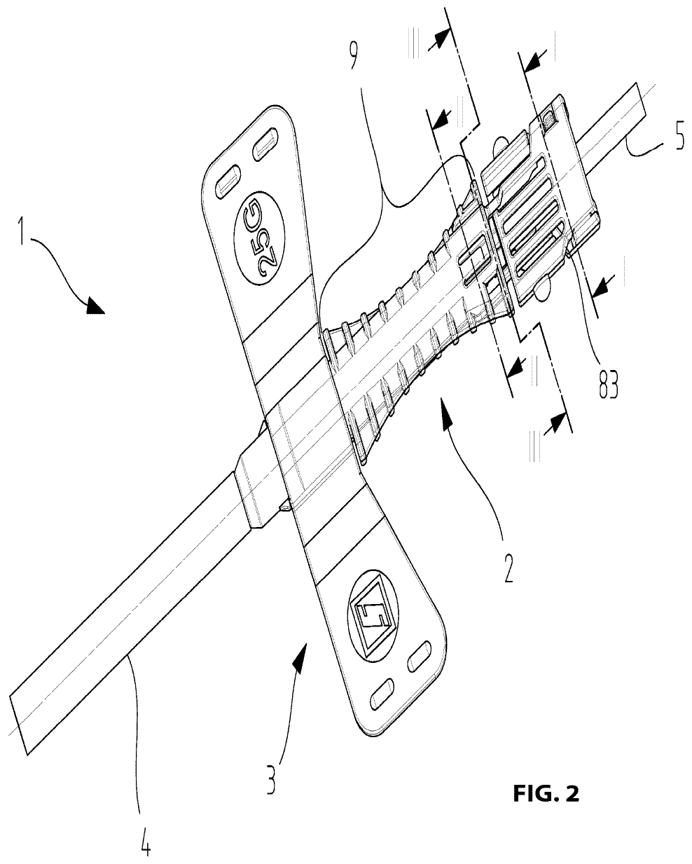

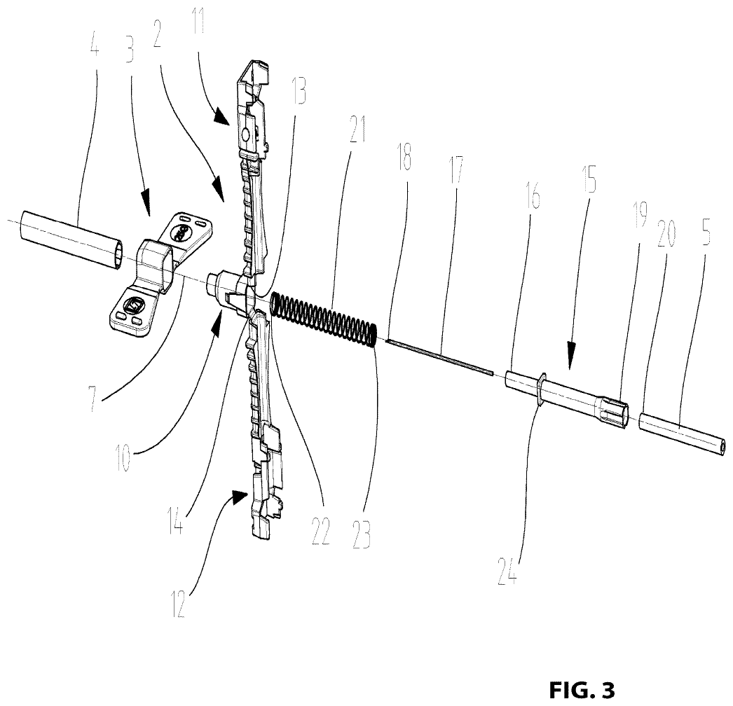

FIGS. 1 and 2 show a safety cannula assembly 1 in different perspectives in shipped configuration including a main body 2 to having an elongated housing, a lobe module 3 arranged at the main body, a tubular cannula protection 4 slid over a distally protruding cannula and a flexible tube 5 that is cut off for illustration purposes. The cut-off tube 5 that is connected at a proximal end section of a sliding body that is not illustrated in FIGS. 1 and 2 has a length of 5 cm to 30 cm and includes an adapter at a proximal end wherein the adapter establishes connectivity with additional handling devices used for blood drawing. The main body 2 includes a plurality of transversal ribs 6 running perpendicular to a longitudinal axis 7 of the main body or also of the entire safety cannula assembly including the sliding body, wherein a protrusion of the transversal ribs beyond the rounded cuboid base geometry of the grip portion 9 of the main body 2 initially decreases from a maximum protrusion at the transversal ribs 6 adjacent to the lobe module 3 towards the proximal end of the grip portion of the main body 2 and subsequently increases again. The transversal ribs 6 running equidistant and parallel to one another are connected with one another and stabilized at each side by a longitudinal rib 8 arranged in a symmetry plane of the main body 2. This way a particularly safe and ergonomic grip is facilitated for two fingers of a person using the safety cannula arrangement 1 wherein the two fingers grip the grip portion 9 of the main body from opposite sides which facilitates handling.

It is evident from the exploded view according to FIG. 3 that the main body 2 is assembled from three main body parts 10, 11 and 12. Thus, from a sleeve shaped first main body part 10 which extends the most in the distal direction and two half shell shaped main body parts including the second main body part 11 and the third main body part 12. The second main body part 11 and third 12 are respectively connected with the first main body part 10 by a film hinge 13, 14, and thus pivotally movable about a respective hinge axis of the film hinge 13, 14 relative to the first main body part 10. The two main body parts 11 and 12 are identically configured but arranged symmetrical relative to a symmetrical plane which is parallel to the two film hinges 13, 14 and runs through the longitudinal axis 7 of the main body.

The inner cavity of the main body 2 includes an essentially hollow cylindrical sliding body 15 that is movably supported in the axial direction, this means in the direction of the longitudinal axis 7 relative to the main body 2. A cannula 17 including a tip 18 formed at a distal end by a ground surface is inserted into the sliding body and sealed therein. A proximal end section 19 that is formed as a socket section is inserted with a distal end section 20 of the tube 5 which is sealed therein. Therefore, the sliding body 15, the cannula 17, and the tube 5 form a firmly connected unit whose components besides the flexibility of the tube 5 are not movable relative to one another. An activation element 21 configured as a coil spring is externally slid onto the distal end section 16 of the sliding body 15 in order to support the sliding body 15 in the operating position under a preload in the main body 2 and in order to provide a propulsion for a pullback movement of the sliding body 15 relative to the main body 2 in the proximal direction, wherein the coil spring is supported with a distal end 22 in the first main body part 10 and supported with a proximal end 23 at a flange 24 that has a rectangular cross section and that protrudes beyond an outer enveloping surface of the sliding body 15.

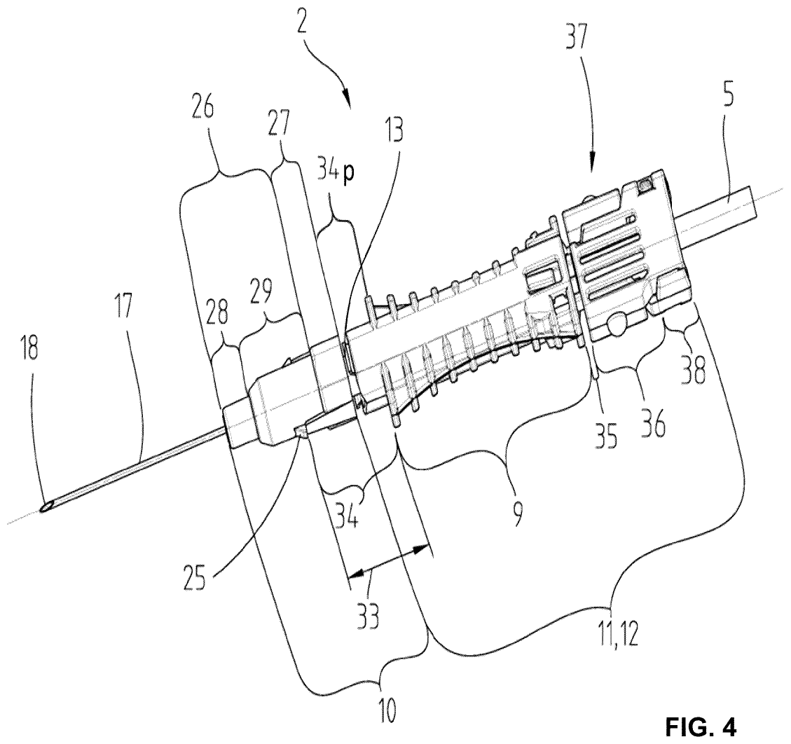

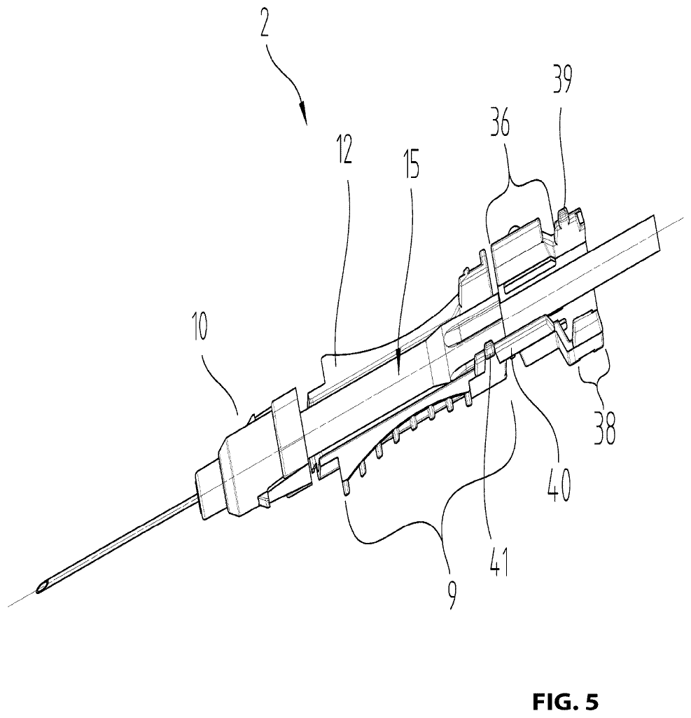

Subsequently the configuration of the main body 2 made from the main body parts 10-12 and the cooperation of the main body 2 with the sliding body 15 is described in more detail with reference to FIGS. 4 through 10.

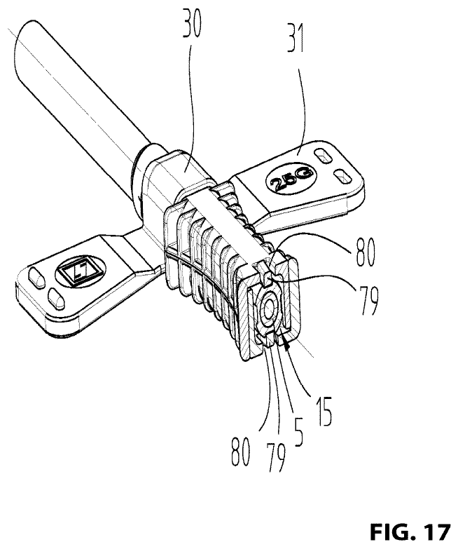

It is evident from FIG. 4 that the main body 2 can be divided into different portions in the axial direction. Starting at a distal end of the main body 2 the sleeve shaped first main body part 10 includes a rotation section that is rotation symmetrical besides two interlocking lugs 25 for fixing the lobe module 26 and an approximately cuboid section 27 adjacent in the proximal direction. The rotation symmetrical section 26 is divided into a cylindrical tip section 28 onto which the cannula protector 4 not illustrated in FIG. 4 but illustrated in FIGS. 1 and 2 can be slid and a cylindrical transition section 29 that has a conical taper at a distal end and that includes a distal end section 16 of the sliding body 15 including an actuation element 21 slid thereon. As evident from FIGS. 3, 1 and 2 the lobe module 3 is slid onto the main body 2 from a distal end of the main body with a center connection part 30 of the lobe module 3 where a respective gripping lobe 31 is integrally provided at opposite longitudinal sides of the lobe module 3 as evident from FIG. 17. An axially measured length 32 of the connection part 31 of the lobe module 3 thus corresponds to a length 33 of an overlapping portion 34 that extends over the first main body part 10 and also over the joined second main body part 11 and the third main body part 12 and that can be divided into the sections 34d and 34p wherein the first distal section 34d overlaps the first main body part 10 and the proximal section 34p overlaps the second and the third main body part 11, 12. The overlapping portion 34 includes an entirety of the cuboid section 27 in the portion of the first main body part 10 and includes a portion of the substantially cylindrical transition section 29, thus up to the interlocking lugs 25 included therein. The connection part 30 of the lobe module 3 thus bridges in particular also the portion of the two film hinges 13, 14 and a gap portion included therein between the first main body part 10 and the second main body part 11 and the third main body part 12. Starting from a proximal end of the overlapping portion 34 the grip portion 9 described supra adjoins in the proximal direction wherein the grip portion is jointly formed by the two main body parts 11 and 12. A circumferential notched portion 35 is arranged further in the proximal direction and a trigger portion 36 adjoins further in the proximal direction and includes a trigger mechanism 37 that will be described in more detail infra. A proximal end section of the main body 2 is formed by a closure portion 38 wherein the second main body part 11 and the third main body part 12 are connected with one another by snap hooks by positive form locking. An opening of the main body 2 provided by a pivot movement of the two main body parts 11 and 12 is thus prevented on the one hand side by the snap hooks in the closure portion 38 and on the other hand side by the connector 30 of the lobe module 3 that is slid over.

FIG. 5 shows how the sliding body 15 is arranged in the interior cavity of the main body 2 not showing the second main body part 11 but only the lower third main body part 12. The distal end section 16 of the sliding body 15 and the actuation element 21 are not shown because they are arranged covered in the sleeve shaped first main body part 10. The closure portion 38 shows a snap hook 39 that cooperates with an accordingly adapted recess in the second main body part 11 in a joined condition of the two main body parts 11 and 12 and that forms a closure that cannot be disengaged without destruction. The trigger portion 36 and the grip portion 9 are exclusively connected by a connection bar 40 in each of the two main body parts 11, 12 wherein the connection bar is connected with its proximal end with the grip portion 9 and connected with the trigger portion 36 at its distal end. It is appreciated that the entire second and third main body part 11, 12 and the main body 2 made from the three main body parts 10, 11, 12 including the film hinges 13, 14 is produced as one integral injection molded component. FIG. 5 shows an additional snap hook 41 at the main body part 12, wherein the snap hook 41 is arranged in the grip portion 9 and also cooperates with an accordingly adapted recess in the second main body part 11 and keeps the two main body parts 11 and 12 together in addition to the snap hooks 39 and the connector 30 of the lobe module 3.

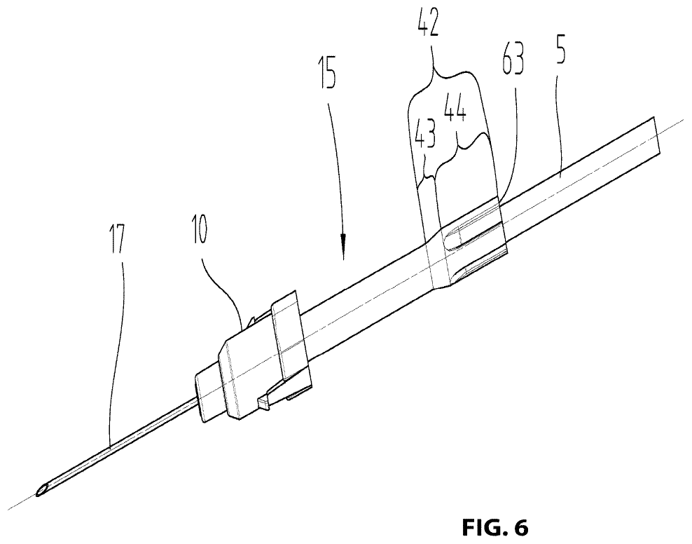

FIG. 6 shows the sliding body 15 including the cannula 17 inserted therein and the tube 5 inserted in the proximal end additionally showing only the first main body part 10. The sliding body 15 includes a socket section 42 that has a greater diameter than the center section and which adjoins in the proximal direction wherein the socket section 42 is formed by a shorter transition section 43 and a substantially cylindrical insertion section 44 adjoining the proximal direction. The insertion section 44 includes an inner cylindrical bore hole that is adapted to an outer diameter of the flexible tube 5 and into which the tube is glued, tightly sealed.

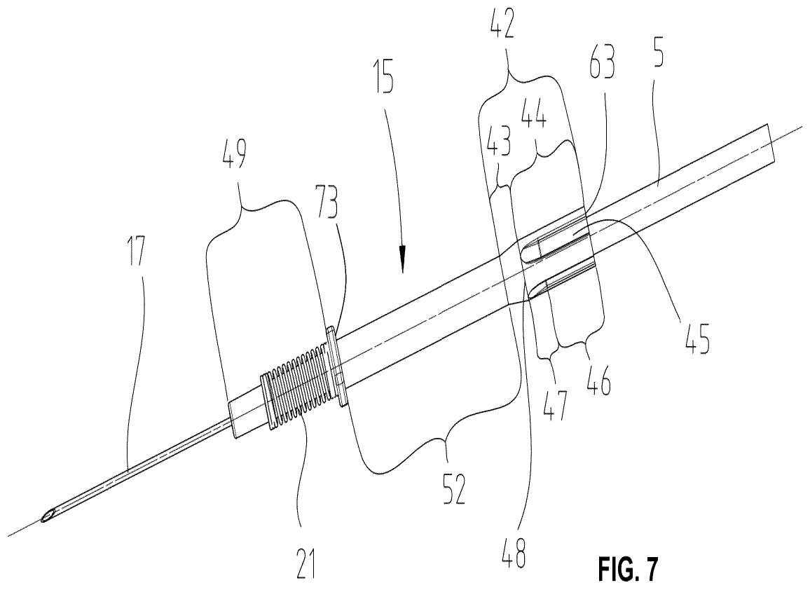

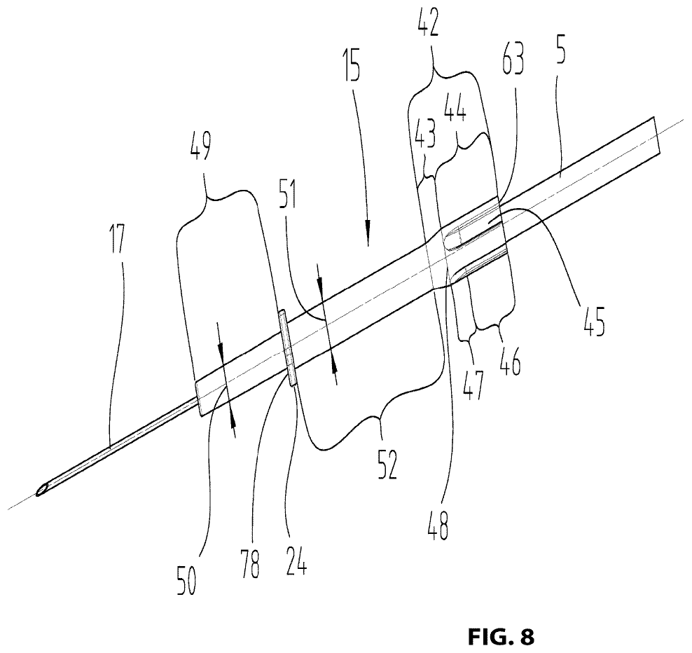

It is evident from FIG. 7 that the socket section 42 includes four stress relief grooves evenly spaced over the circumference of the socket section 42 and running in a direction of the longitudinal axis of the sliding body 15 for blocking tongues of the main body that will be described infra. The stress relief grooves 45 include a longer section 46 where they have a greater depth and a groove base that runs parallel to the longitudinal axis 7 and a shorter distal section 47 where the groove base rises in a ramp to a circumferential border line between the insertion portion 44 and the conical transition section 43. The function of the stress relief grooves in combination with the blocking tongues recited supra will the described infra.

FIG. 7 illustrates the compressed actuation element 21 on a distal end section 49 of the sliding body 15 which is not the case in the otherwise analogous representation of FIG. 8. This indicates that a diameter 50 in the distal end section 49 of the sliding body 15 is greater than a diameter 51 in a center section 52 of the sliding body 15.

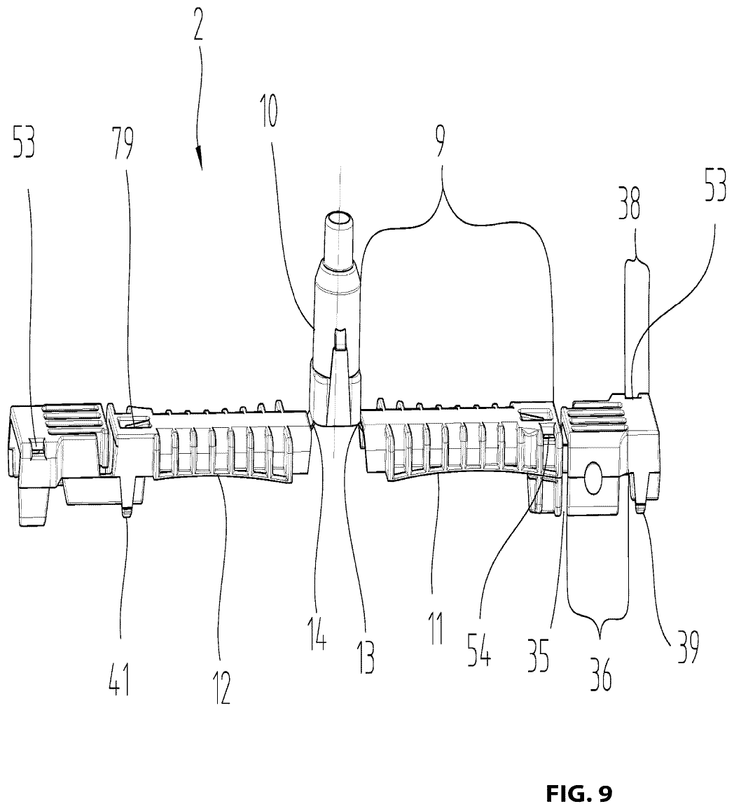

The individual portions of the main body 2 and the mirror symmetrical arrangement of the identically formed main body parts 11 and 12 are evident from FIG. 9 where the main body 2 is shown with the main body parts 11 and 12 laterally folded out from the first main body part 10. In this condition the main body 2 is removed as an injection molded component from the injection molding tool. The grip portion 9, the adjacent notch portion 35, the adjoining trigger portion 36 and the closure portion 38 forming the proximal closure are evident from FIG. 9. A recess 53 is visible on the third main body part 12, shown on the left side of FIG. 9, wherein the snap hook 39 is receivable and lockable in the recess 53 as evident from FIG. 5. Another recess 54 in the grip portion 9 is shown and provides locking reception of the corresponding snap hook 41 at the grip portion 9 of the third main body part 12. Thus, there is a total of two snap hooks 39 and two recesses 53 in the closure portion 38 and two snap hooks 41 and two recesses 54 in the grip portion 9. Each of the two main body parts 11 and 12 includes a respective snap hook 39, 41 and a respective recess 53, 54 in the grip portion 9 as well as in the closure portion 38.

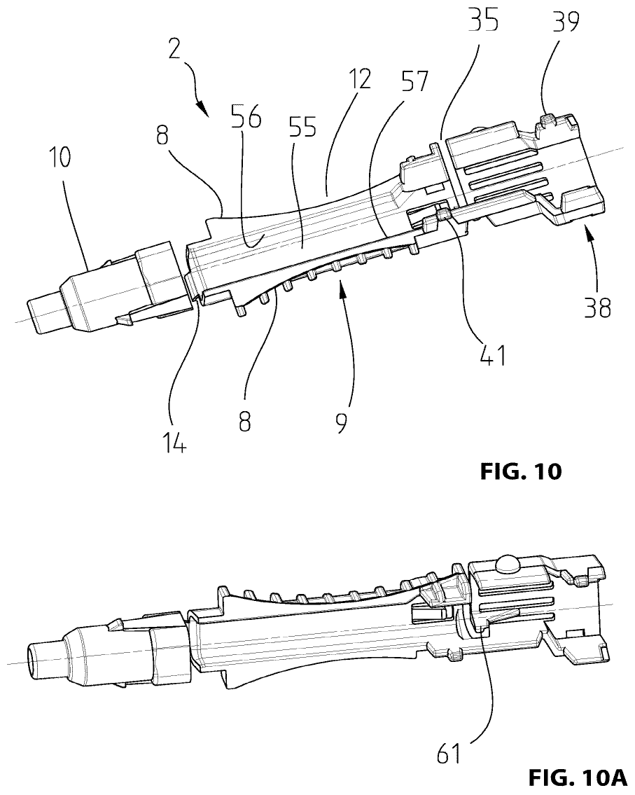

FIG. 10 shows a view into the groove shaped third main body part 12 since the second groove shaped main body 11 part was removed which would otherwise be arranged there above in an assembled condition and on the other hand side also the sliding body 15 and the cannula 17 and the tube 5 are not inserted. It is evident that a base 55 and two opposite half-walls 56, 57 of the main body part 12 are arranged perpendicular to one another which yields a square cross section of the interior cavity when the two main body parts 11, 12 are assembled. The two longitudinal ribs 8 of the main body part 12 arranged on opposite sides are jointly formed by both main body parts 11 and 12 in assembled condition and define a center plane in a contact portion of the two main body parts 11, 12 wherein the center plane runs parallel to the longitudinal axis 7 and also forms a plane of symmetry. FIG. 10 also shows the snap hooks 41 in the grip portion 9 and 39 in the closure portion 38 wherein the snap hooks are arranged on opposite sides in respect to the longitudinal axis 7

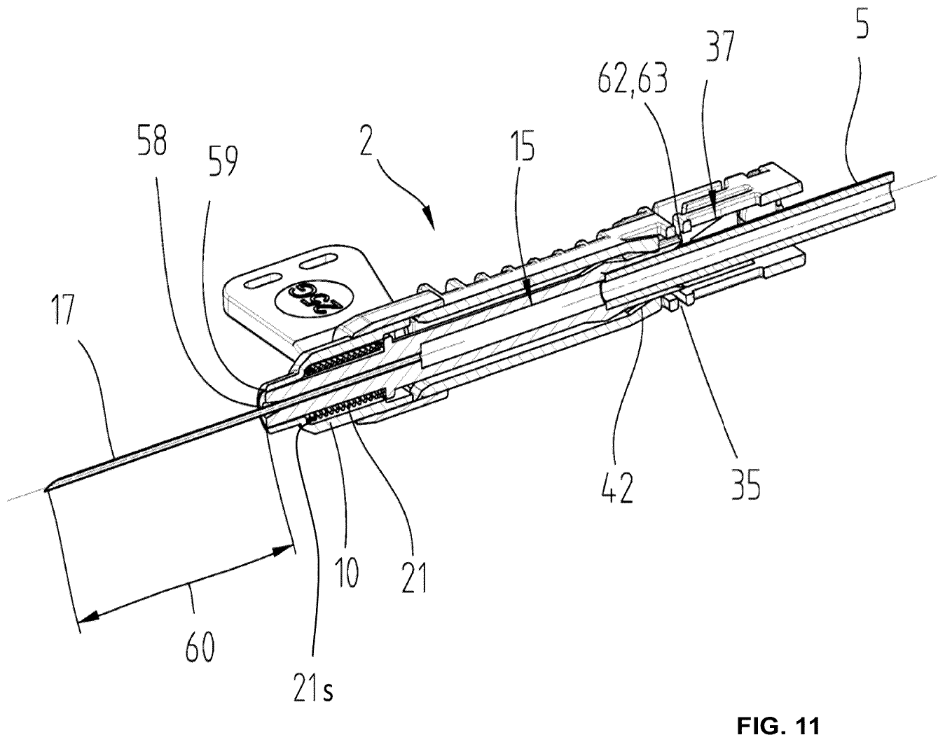

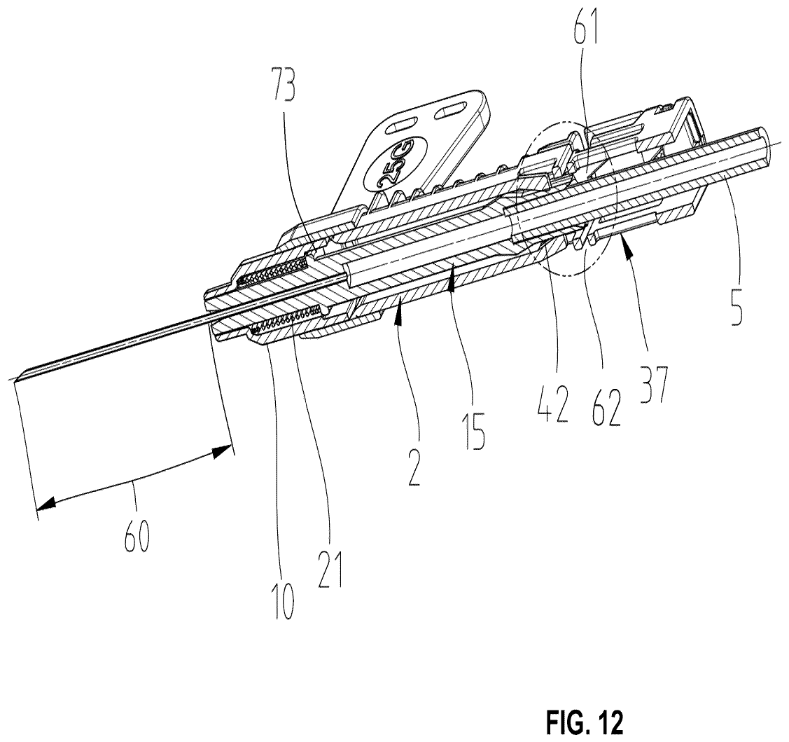

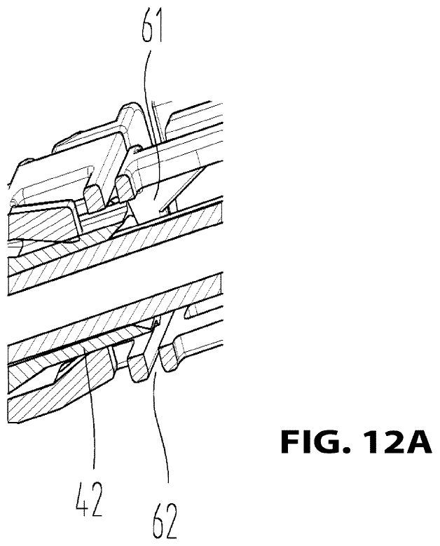

FIGS. 11, 12 and 12a respectively show a longitudinal sectional view of the safety cannula arrangement 1 in an operating condition of the sliding body 15 in different perspectives. A distal face 58 of the sliding body 15 thus eventually terminates flush with a distal face 59 of the first main body part. The cannula 17 protrudes with a free length 60 beyond the distal face 59 of the base body part 10. The actuation element 21 is in a preloaded condition so that the sliding body 15 tends to move relative to the main body 2 in the proximal direction. This movement is prevented by two locking elements 61a, 61b which form part of the trigger mechanism 37 and cooperate with a proximal face 62 of the sliding body 15 in its socket section 42. The face 62 of the sliding body 15 forms a shoulder 63 in combination with the tube 5 that has a smaller diameter which is evident from FIGS. 6 through 8. This shoulder is provided in the operating position 15 viewed in the axial direction in the notched portion 35 at its proximal end that is defined by the locking elements 61a, 61b of the trigger mechanism 37. The sliding body 15 is thus fixed without clearance in the axial direction in the main body 2 in the operating position which is indispensable for performing the puncture correctly.

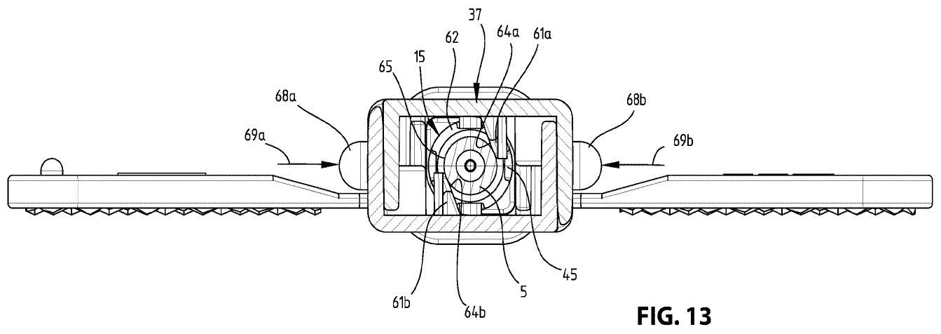

Viewing FIGS. 13 through 16 and 18 together makes the function of the trigger mechanism 37 evident. The proximal face 62 that forms the shoulder 63 in the transition from the tube 5 to the socket section 42 of the sliding body 15 is retained by two diametrically opposed locking elements 61a, 61b, c.f. also FIG. 10a, so that the sliding body 15 remains in the operating position 45. The socket section 42 of the sliding body 15 includes relief grooves which yields a circular contour of the face 62 in a cross section, wherein the circular contour is interrupted by four indentations having a 90-degree offset.

The locking elements 61a, 61b have a triangular or trapezoid shape in an axial viewing direction and contact an outer enveloping surface 65 of the tube 5 with a respective control edge 64a, 64b.

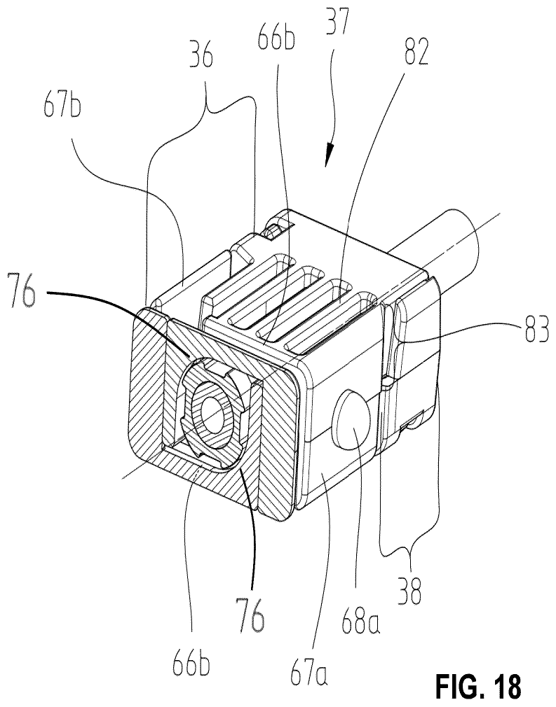

It is evident from FIG. 18 that the locking elements 61a, 61b are coupled respectively by a compression rod 66a, 66b with a trigger member 67a, 67b. The trigger members 67a, 67b are respectably provided with a protrusion 68a, 68b configured as a semi spherical button. The trigger members 67a, 67b form two approximately square surfaces in a side view of the main body 2, the square surfaces having a square protrusion 68a, 68b wherein the compression rods 66a, 66b run at an angle of 90 degrees relative thereto. Thus, the locking elements 61a, 61b run at an angle of approximately 90 degrees relative to the compression rods 66a, 66b so that a C-shape or U-shape is achieved overall. Thus, the trigger members 67a, 67b and the locking elements 61a, 61b respectively form a free arm of the U or C, whereas the compression rods 66a, 66b form a base element of the U or C.

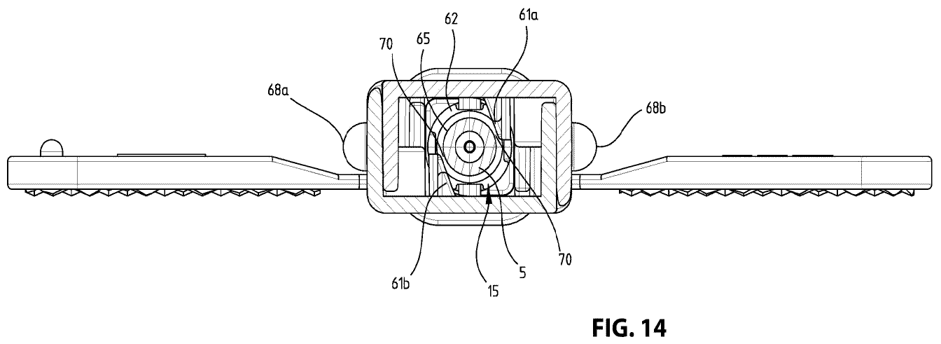

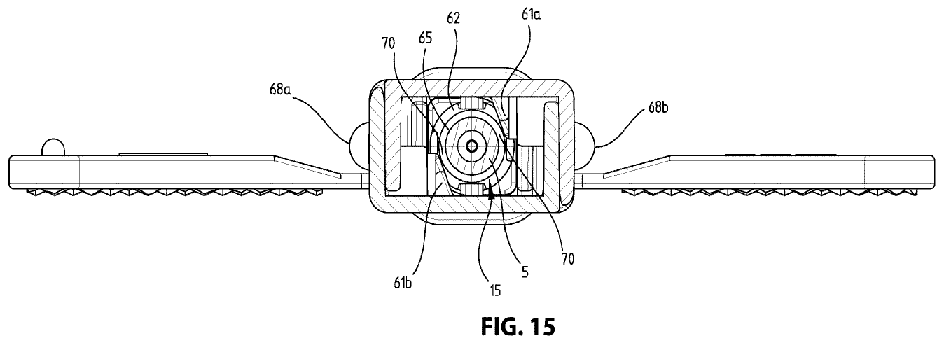

When the sliding body 15 needs to be transferred into the safety position after completing the blood draw or the infusion with the safety cannula arrangement 1 so that the cannula 17 is arranged within the main body 2 in its entirety, the operator of the safety cannula arrangement 1 imparts a pressure force upon the protrusions 68a, 68b of the trigger members 67a, 67a wherein the pressure force is imparted with two fingers of one hand simultaneously and runs in a radial direction according to the arrows 69a, 69b. Starting from the locking position illustrated in FIG. 13 where the locking elements 61a, 61b block the sliding body 15 by an engagement (contact at the face 62) so that the upper locking element 61a in FIG. 13 moves to the right due to pressure upon the left trigger member 68a whereas the lower locking element 61b in FIG. 13 moves to the left due to pressure upon the right trigger member 68b. FIG. 14 shows a condition where a distance 70 has formed between the locking elements 61a, 61b and the outer enveloping surface 65 of the tube 5 due to a displacement of the trigger members 68a, 68b forming a rigid unit and of the locking elements 61a, 61b connected therewith. This distance 70, however, is not large enough yet to release a contact of the face 62 at the locking elements 61a, 51b. Therefore, the cannular carrier 15 remains in the operating position in this position of the trigger mechanism or the trigger members 67a, 67b. FIG. 15 shows another intermediary state where the distances 70 between the locking elements 61a, 61b and the enveloping surface 65 of the tube 5 are larger but still cause an overlap between the locking elements 61a, 61b and the face 62 of the sliding body 15 and thus a blocking of the sliding body 15.

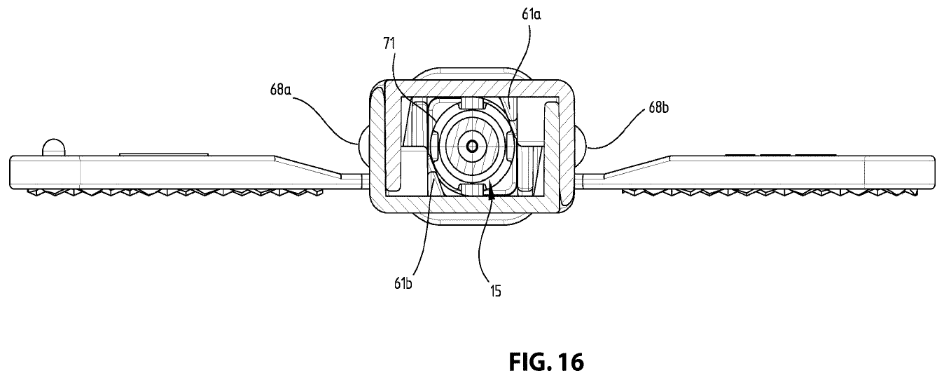

When the trigger members 67a, 67b with their associated knob shaped protrusions 68a, 68b are in the position shown in FIG. 16 the associated locking elements 61a, 62b are displaced outward enough so that the cross section of the sliding body 15 defined by a circular enveloping line 71 is released in its entirety in the portion of the socket section 42. Now, the locking elements 61a, 61b are out of engagement with the sliding body 15 so that the sliding body 15 can move in the proximal direction driven by the actuation element 21 which causes a pull back of the cannula 17 into the interior of the main body 2.

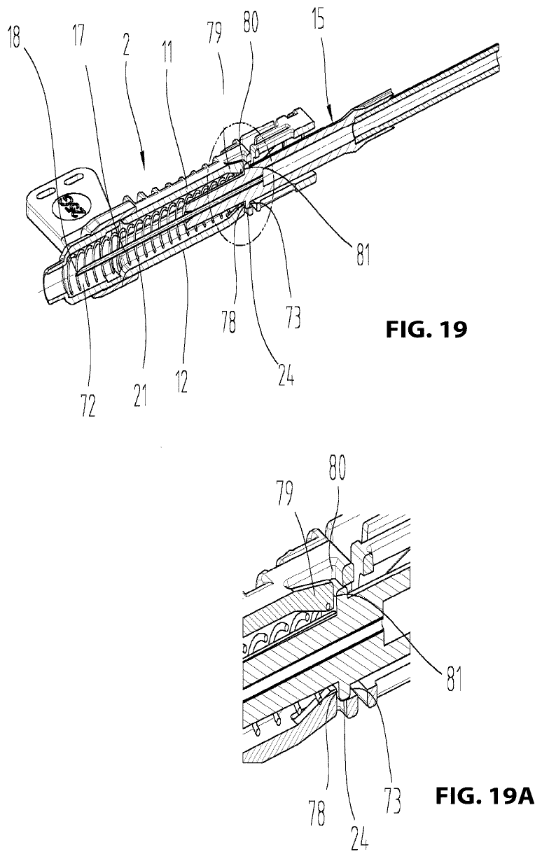

Accordingly, FIGS. 19 and 19a show the sliding body 15 in the safety position where the cannula 17 including the tip 18 is arranged within an interior cavity 72 of the main body 2. The actuation element 21 configured as the coil spring is now provided in a condition where it has less tension than in the operating position of the sliding body 15.

In order to prevent an exit of the entire sliding body 15 and thus also of the cannula 17 itself at the proximal end of the main body 2 the safety position of the sliding body 15 is defined by a stop surface 73 arranged at the sliding body 15. The stop surface 73 arranged at the sliding body 15 thus cooperates with a stop surface 74 of the main body 2 that is formed at the proximal end of the main body 2, thus at the proximal end of the closure portion 38 which is formed jointly by the second main body part 11 and the third main body part 12.

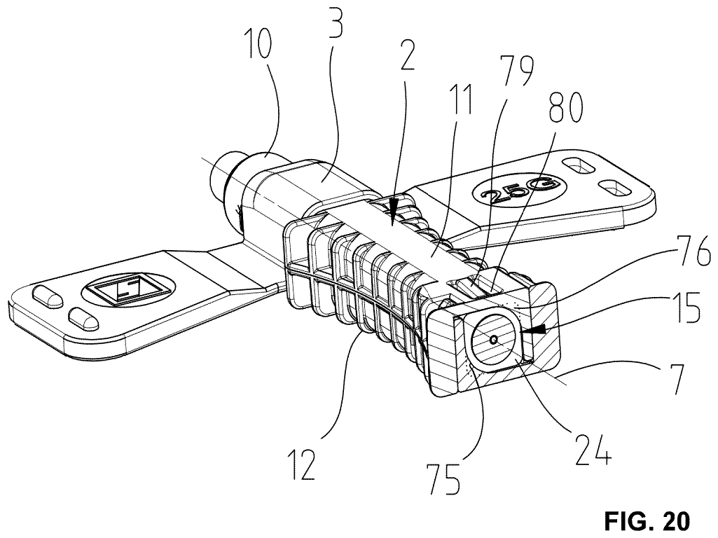

It is evident from the sectional view of FIG. 20 that the second main body part 11 and the third main body part 12 are U-shaped in cross section wherein the two U-shaped cross sections of the walls are nested into one another in a direction perpendicular to the plane of symmetry of the main body 2, this means in a direction perpendicular to the two film hinges, this means of the pivoting motion during the joining process and thus define an essentially square free cross section in the interior cavity 72 of the main body 2. The cross-section shape in the portion of the stop surfaces 74 of the main body parts 11 and 12, however is not exactly square but two corner portions are filled and defined towards the longitudinal axis 7 of the main body 2 respectively by an arcuate line 75 which forms a quarter circle in the respective corner of the U or C. FIG. 20 shows the edges of an imaginary square where the stop segments 76 defined by the arcuate line 75 are introduced into the corners in dashed lines for the purpose of clarity.

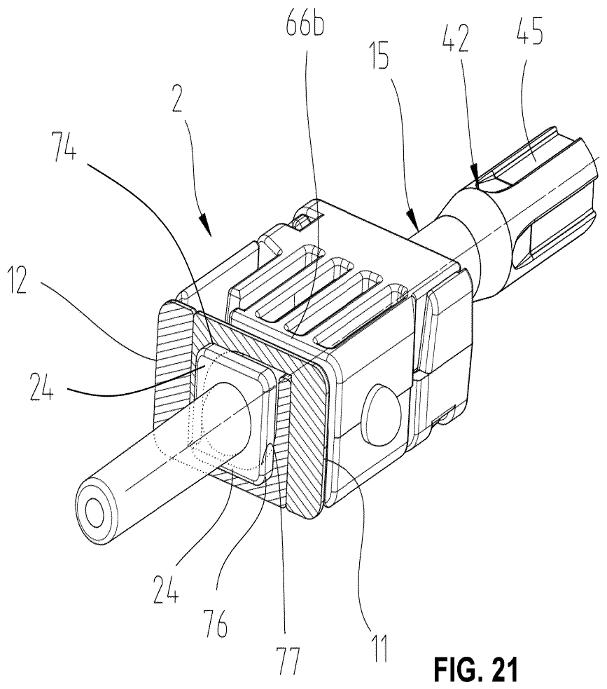

As evident from FIG. 20, the radius of a circle that runs through the slightly rounded corners of the square flange 24, is greater viewed in the direction of the longitudinal axis 7 than a radius of a circle which is formed by a continuation of the two arcuate lines 75. Therefore, the square flange 24 cannot pass through the free cross section in the area of the stop surfaces 74 that are formed at the stop segments 76, so that a displacement of the sliding body 15 stops at this location. This is also clearly evident from the sectional view according to FIG. 21. The square flange 24 contacts the stop surfaces 76 of the main body parts 11, 12 in the portion of the rounded sections 77. The cannula carrier and sliding body 15 is in the safety position in the condition illustrated in FIG. 21 where the socket section 42 has exited from the main body 2 at a proximal end of the sliding body 15.

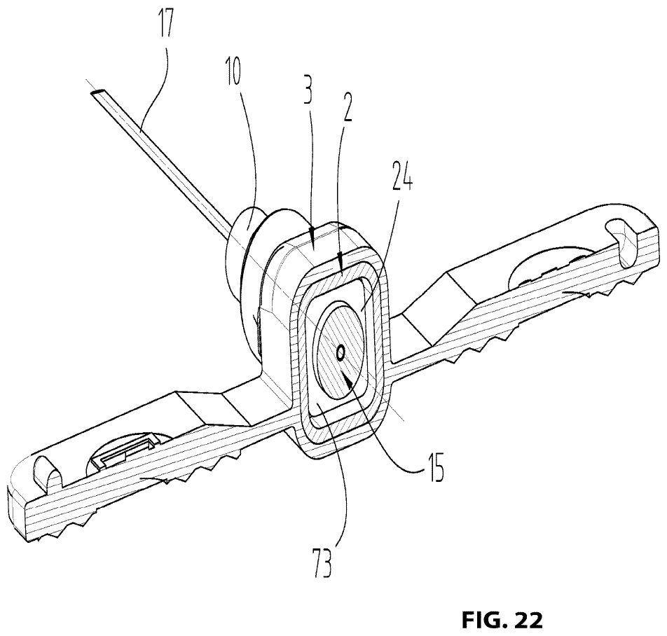

It is evident from FIG. 22 that the interior cavity 72 in the cuboid section 27 of the overall sleeve shaped first main body part 10 is square with rounded corners. The cross section of the square flange 24 is adapted to this cross-section shape so that a smooth low resistance movement of the sliding body 15 is facilitated in the axial direction under an impact of the force of the actuation element 21 and a rotation of the sliding body 15 about the longitudinal axis 7 is reliably prevented. Thus, the flange 24 performs the function of a rotation blocking body of the sliding body 15 and the corresponding cuboid section 27 of the first main body part 10 forms a rotation blocking body of the main body 2. While the proximal contact surface 73 is arranged at the square flange 24 of the sliding body 15 defining the pull back movement of the sliding body 15 the square flange 24 has a double function since its additional stop surface 78 that is not shown in FIG. 22, but shown in FIGS. 7 and 8 supports the actuation element 21.

In addition to limiting the pull back movement of the sliding body 15 in the proximal direction it is also important to prevent that the sliding body 15, after having moved into the safety position, is moved back in the distal direction intentionally or unintentionally so that the cannula 17 is exposed again and the safety cannula arrangement 1 may be used again which shall be avoided and prevented under all circumstances. The blocking tongues 79 recited supra serve this purpose wherein the blocking tongues are configured as spring elastic elements of the main body 2 and respectively arranged within a window 80 enveloping the blocking tongues 79 on three sides. This configuration is clearly evident from FIGS. 19 and 19a, where the sliding body 15 is in the safety position. A respective face 81 of the blocking tongues 79 contacts the distally oriented contact surface 78 of the square flange 24 so that the square flange 24 performs three functions: end stop for the pull back movement, support of the actuation element 21 and stop surface for the blocking tongues 79. Since the blocking tongues 79 are elastically connected with the respective main body part 11, 12 the blocking tongues 79 move elastically radially outward when the sliding body 15 passes through during the pull back movement and move back radially inward after the passage of the square flange 24 due to the preload of the blocking tongues 79 and perform the blocking in cooperation with the flange 24. Since the diameter of the sliding body 15 in the distal end section 49 is smaller than in the center portion 52 a sufficiently large overlap of the blocking tongues 79 with the protrusion of the flange 24 beyond the adjacent cylindrical surface is provided viewed in radial direction.

As evident from FIG. 9 the blocking tongues 79 are formed during injection molding of the main body parts 11, 12 so that they are inclined relative to the longitudinal axis 7 of the main body 2 or the sliding body 15 starting from a connection cross section with the associated main body part 11, 12 and moving towards their free ends. In order to maintain this preload radially inward reliably even during a long storage time of the safety cannula arrangement 1 relief grooves 45 are formed in the socket section 42 of the sliding body 15 as described supra. The relief grooves facilitate an orientation of the blocking tongues 79 that is inclined relative to the longitudinal axis 7 during storage in the operating condition of the sliding body exactly in the shape of the relief tongues that is subsequently required in the safety position of the sliding body 15 to block another deployment movement of the sliding body 15. This way material fatigue and a loss of the radially inward oriented preload is prevented compared to a configuration of the socket section 42 without the relief grooves 45 which could otherwise lead to a failure of the blocking tongues 79 so that a redeployment of the cannula 17 would not be prevented reliably.

The following is appreciated regarding the configuration of the trigger mechanism 37 with reference to FIG. 18, as well as FIGS. 10, 10a and 4.

As stated supra, the main body 2 is substantially cuboid in the trigger portion 36, wherein a respective trigger member 67a, 67b and the associated compression rod 66a, 66b in combination with link elements configured as bar 82 jointly form a L-shaped wall arrangement, wherein the three link elements are connected by the respective compression rod 66a, 66b. Due to the small cross sections of the bars 82, they can be deformed easily by pressure upon the trigger members 67a relative to the proximal closure portion 38. When pressure is imparted upon the trigger members 67a, 67b, a connection cross section 83 arranged between the bigger members 67a, 67b and the closure portion 38 functions as an additional pivot joint. A trigger surface respectively formed by the trigger member 67a, 67b runs at an angle of 90 degrees relative to a plane in which the bars 82 and the respective compression rod 66a, 66b connecting the bars 82 are arranged. The respective locking elements 61a, 61b extend at an angle of 90 degrees relative the plane of the bars 82, and the respectively associated compression rod 66a, 66b. Overall, a C- or U-arrangement is achieved in cross section.

### REFERENCE NUMERALS AND DESIGNATIONS

1 safety cannula arrangement
2 main body
3 lobe module
4 cannula protector
5 tube
6 transversal rib
7 longitudinal rib
8 longitudinal rib
9 grip portion
10 first main body part
11 second main body part
12 third main body part
13 film hinge
14 film hinge
15 sliding body
m enveloping surface
16 distal end section
17 cannula
18 tip
19 proximal end section
20 distal end section
21 actuation element
21s support surface
22 distal end
23 proximal end
24 flange
   25 interlocking lug
   26 rotation symmetrical section
   27 cuboid section
   28 tip section
   29 transition section
   30 connection part
   31 gripping lobe
   32 length
   33 length
   34 overlapping portion
   34d section
   34p section
   35 notched portion
   36 trigger portion
   37 trigger mechanism
   38 closure portion
   39 snap hook
   40 connection bar
   41 snap hook
   42 socket section
   43 transition section
   44 plug in section
   45 stress relief groove
   46 section
   47 section
   boundary line
   49 end section
   50 diameter
   51 diameter
   52 center portion
   53 recess
   54 recess
   55 base
   56 semi-wall
   57 semi-wall
   58 face
   59 face
   60 free length
   61a, 61b locking element
   62 proximal face
   63 shoulder
   64a, 64b control edge
   65 enveloping surface
   66a, 66b compression rod
   67a, 67b trigger member
   68a, 68b protrusion
   69a, 69b arrow
   70 distance
   71 circumferential line
   72 inner cavity
   73 stop surface
   74 stop surface
   75 arcuate line
   76 stop element
   77 section
   78 stop surface
   79 blocking tongue
   80 window
   81 face
   82 bar
   M 83 connection cross section
   84 rotation blocking device

What is claimed is:

1. A safety cannula assembly, comprising:
a cannula configured to puncture human or animal tissue, wherein the cannula includes a distal end portion including a tip;
a sliding body including a distal end portion connected to the cannula and a proximal end portion connected to a flexible tube, wherein a flow connection extends through the sliding body between the tip of the cannula and a proximal end of the flexible tube;
a main body in which the sliding body is displaceable from an operating position, where the tip of the cannula is located outside the main body to a safety position, where the tip of the cannula is located inside the main body, wherein a stop surface of the sliding body contacts a stop surface of the main body in the safety position, so that further axial displacement of the sliding body relative to the main body in the proximal direction is prevented;
an actuation element arranged between the main body and the sliding body and configured to displace the sliding body from the operating position to the safety position; and
a trigger mechanism arranged at the main body and configured to trigger the displacement of the sliding body from the operating position to the safety position, wherein the trigger mechanism includes at least one trigger member loadable by a radially oriented compressive force or loadable with a torque relative to the main body about a longitudinal axis of the main body, and a locking element displaceable from a locking position, where the locking element engages the sliding body and locks the sliding body in the operating position, to a trigger position, where the locking element is disengaged from the sliding body so that the sliding body moves into the safety position;
a rotation blocking device which prevents a rotation of the sliding body arranged in the operating position about its longitudinal axis inside the main body, wherein the rotation blocking device includes at least one rotation blocking body of the sliding body and at least one rotation blocking body of the main body that cooperates with the at least one rotation blocking body of the sliding body,
wherein the stop surface of the sliding body is arranged at the at least one rotation blocking body of the sliding body.

2. The safety cannula assembly according to claim 1, wherein the sliding body is configured rigid overall.

3. The safety cannula assembly according to claim 2, wherein the sliding body has the same shape and/or the same configuration and/or the same contour in the operating position as well as in the safety position as well as in all intermediary positions.

4. The safety cannula assembly according to claim 3, wherein the main body includes at least one gripping lobe or two respective gripping lobes at two opposite sides.

5. The safety cannula assembly according to claim 4,
wherein a grip portion configured for handling the safety cannular assembly during puncture is exclusively arranged at the main body, and
wherein ribs or a plurality of protrusions configured to improve gripping are advantageous arranged in the grip portion.

6. The safety cannula assembly according to claim 1,
wherein the sliding body is configured as a hollow cylinder and the rotation blocking body of the sliding body is a protrusion or a flange that protrudes radially outward beyond an outer enveloping surface of the hollow cylinder, and
wherein the protrusion or flange has a square contour viewed in the direction of the longitudinal axis of the sliding body wherein corner portions of the square contour are rounded.

7. The safety cannula assembly according to claim 1,
wherein the main body includes an interior space with a square cross section in a section forming the rotation blocking body of the main body, and
wherein the section forming the rotation blocking body of the main body forms a one-piece sleeve body or a portion of a one-piece sleeve body.

8. The safety cannula assembly according to claim 1, wherein the actuation element is supported at a distal face of the rotation blocking body of the sliding body sliding body and oriented opposite to the stop surface of the sliding body.

9. The safety cannula assembly according to claim 1, wherein the stop surface of the main body is formed by at least one protrusion that extends radially inward from a wall of the main body forming the interior cavity of the main body.

10. The safety cannula assembly according to claim 9,
wherein two protrusions are provided that are arranged diametrically opposed to one another and respectively shaped approximately triangular viewed in the direction of the longitudinal axis of the sliding body, and
wherein one side of the triangle is advantageously rounded concave, wherein each protrusion connects two wall sections of the main body in a corner portion, wherein the two wall sections are arranged L-shaped relative to each other in cross section.

11. The safety cannula assembly according to claim 10, wherein the two protrusions contact an outer enveloping surface of the sliding body in the operating position of the sliding body and prevent a radial displacement of the sliding body in all radial directions.
